(19) United States
(12) Patent Application Publication
(10) Pub. No.: US 2007/0123858 A1
(43) Pub. Date: May 31, 2007

(54) External fixation device for osteosynthesis

(76) Inventors: Beat Strub, Kappel (CH); Carlos Satizabal, Bogota (CO); Martin Munch, Liestal (CH)

Correspondence Address: JONES DAY, 222 EAST 41ST STREET, NEW YORK, NY, 10017-6702, US

(21) Appl. No.: 11/519,195

(22) Filed: Sep. 8, 2006

Related U.S. Application Data
(63) Continuation of application No. PCT/IB04/00674, filed on Mar. 10, 2004.

(51) Int. Cl. A61B 17/00

(52) U.S. Cl. 606/54

(57) ABSTRACT

An external fixation device having a frame comprising at least one rod which takes up the mechanical loads. Frame and bone are connected via connecting means known per se, in particular bone-retaining pins. These connecting means in turn are connected to the frame via clamping jaws. These clamping jaws permit detachable fixing and arbitrary arrangement and grouping on the frame. A module which can be mounted on the frame is provided. By means of the module, a movement of two bone fragments relative to one another can be effected. It has at least two components. The first of the at least two components can be connected to the frame. The second of the two components can be connected to at least one clamping jaw of a connecting means which is connected to a bone fragment.

### CROSS-REFERENCE TO RELATED APPLICATIONS

This application is a continuation of International Patent Application No. PCT/IB2004/000674, filed on Mar. 10, 2004, the entire contents of which are incorporated herein by reference thereto.

### FIELD OF THE INVENTION

The present invention relates to an-external fixation device and method for osteosynthesis.

### BACKGROUND OF THE INVENTION

Orthopaedic corrections, such as, for example, limb extension, are carried out nowadays, inter alia, using so-called fixation devices. The proximal end of a bone may be connected by means of bone-retaining pins through the skin to an external frame (located outside the body) which, during the extension process, bears the mechanical loads which are normally borne by the bone. The connection between the screws and the frame is realized by means of clamping jaws.

The distal end of the bone is likewise connected to the external frame by means of bone-retaining pins through the skin. A bone fixed in this manner can then be parted by means of an osteotomy (saw-cut) between the proximal and distal screws. All forces which are normally taken up by the bone now pass via the external frame.

After a number of days, the bone begins to form fibrous tissue in an attempt to close the artificially created gap. As soon as the two bone fragments are connected with the fibrous tissue, extension can be started. The distal and proximal fragments are slowly drawn apart (approx. 1 mm per day), allowing the tissue and the bone to grow. When the desired extension is reached, the procedure is stopped and the external fixation device is left in place until the bone has been consolidated and can bear all forces itself.

According to the same principle of operation as in the case of extending a bone, bone segments can also be transported from one part of the bone to the other in order to close a gap which has formed, for example, by the removal of a tumor. The distal and proximal fragments may be held in their anatomically correct position by the frame. Additionally bone-retaining pins are mounted in between the bone fragments and the osteotomy is carried out. A middle segment formed is displaced without changing the total length of the bone. Displacement is stopped when the gap has closed.

A problem exists in that the external frame firstly must be rigid in order to be able to bear forces and secondly must permit a movement of the fragment in a defined axis (parallel to the extension direction). This movement must furthermore be defined as a function of time (for example, 0.25 mm every six hours).

The prior art discloses a special frame which extends telescopically and permits a defined extension by means of an internal spindle drive. This unit must be very exactly constructed and is permitted to have little movement so that the fracture is not excessively stressed. The complicated constructions are often expensive to produce.

Furthermore, a disadvantage of telescopic constructional systems is that the stability of the frame is impaired such that telescopic constructional systems generally have to be made comparatively solid in order to ensure that the bearing forces are reliably taken up.

The prior art furthermore discloses arrangements which differ from the telescopic constructional system of a supporting rail. German document DE 32 29 313 discloses a device whose supporting rails consist of two rigid support parts which in each case are connected to bone-retaining pins of a bone fragment so that a relative longitudinal displacement of the bone fragments is possible. The second supporting part is in the form of an auxiliary rail. The auxiliary rail is displaceable on the main rail, with the result that relative movement of the bone fragments is achieved. This device, too, must be kept relatively solid because there are strong forces carrying out the movement of the bone fragments relative to one another and which have to be dissipated.

Finally, German document DE 36 11 319 adopts another approach for avoiding telescopic rails. In this device, two rods are arranged parallel to each other and form an external support frame. The frame is connected to the bone with the aid of screws or bone-retaining pins. Relative movement of the two bone fragments is now possible by means of movement in the guidance of the screws or bone-retaining pins so that the bone-retaining pins can change their angle relative to the rods and hence to the frame by the movement of the bone. The rods therefore do not change their position relative to one another. Compensation of the movement of the bone fragments is achieved by angling of the bone-retaining pins. Precise guidance and defined changes in length are not possible with this device.

### SUMMARY OF THE INVENTION

It is therefore an object of the present invention to provide an external fixation device for osteosynthesis which avoids the above-discussed disadvantages. The fixation device should be stable and be able to reliably absorb the transmitted forces. Furthermore, the design should be comparatively simple, allowing for easy and simple changes in length.

This object is achieved by an external fixation device having a frame comprising at least one rod which takes up the mechanical loads. Frame and bone are connected via connecting means known per se, for example bone-retaining pins. These connecting means in turn are connected to the frame via clamping jaws. These clamping jaws permit detachable fixing and arbitrary arrangement and grouping on the frame. A module which can be mounted on the frame is provided. By means of the module, a movement of two bone fragments relative to one another can be effected. It has at least two components. The first of the at least two components can be connected to the frame. The second of the two components can be connected to at least one clamping jaw of a connecting means which is connected to a bone fragment.

The module furthermore has a means with which the second component can be moved relative to the first component. As a result, a relative movement is achieved. This ensures that, by means of the clamping jaw to which the second component is connected, the bone fragment connected to the clamping jaw and hence to the second component can be moved.

Owing to the relatively weak mechanical load of the module, the module can be constructed to be much more delicate than a conventional extension apparatus. It is therefore possible to construct the module to be very light, for example of aluminum, so that it can be left on the frame in the static periods too.

The frame is preferably formed from at least two rods arranged parallel. However, other arrangements are also conceivable and in principle the design of a frame having only one rod is possible.

A method of performing osteosynthesis includes the steps of connecting a fixation device via connecting means to two bone fragments. The fixation device may include a frame having at least one rod, a plurality of clamping jaws, the plurality of clamping jaws connecting the connecting means to the frame, and a module mounted on the frame. The module may have at least two components, a first component and a second component, of which the first component connected to the frame and the second component connected to at least one clamping jaw. The module may further include means comprising a hand wheel and threaded rod with which the second component can be moved relative to the first component. The method further includes displacing a bone fragment relative to another by loosening the clamping jaws which hold the bone fragment to be transported such that the clamps can be displaced on the frame turning the hand wheel rotating the threaded rod, permitting a controlled displacement of the clamping jaws along the frame, and tightening all clamping jaws of the fixation device during a static period, wherein the module is ineffective and not under mechanical load.

### BRIEF DESCRIPTION OF THE FIGURES

The invention and further developments of the invention are explained in even greater detail in the following exemplary drawings. The present invention can be better understood by reference to the following drawings, wherein like references numerals represent like elements. The drawings are merely exemplary to illustrate certain features that may be used singularly or in combination with other features and the present invention should not be limited to the embodiments shown.

FIG. 1 shows a perspective diagram of an external fixateur according to the invention and

FIG. 2 shows a schematic perspective diagram of a fixateur module according to the invention.

### DETAILED DESCRIPTION

The fixation device based on a traditional frame having simple, non-telescopic elements. Rods may be used and permit a modular design of various frames. A preferred design is a parallel arrangement of two rods so that a double rail forms. The frame is oriented so that it points in the direction of displacement. Such a frame is provided with clamping jaws on which the bone-retaining pins are fixed. The forces pass via the immobile frame without mechanical displacement units. The actual invention is an additional device, designated as module herein, which is preferably mounted only temporarily to effect an extension increment and, if required, can be removed again at any time without having to remove the entire fixation device.

The time scheme for an extension procedure may consist of a short extension time (approx. 2 minutes) and a long static time (approx. 6 hours). During the static time, all clamping jaws of the fixation device are tightened and the module is ineffective and not under mechanical load. Stability is ensured in an operationally safe manner by the simple frame. In the short extension phase, the bone is brought into a position without load. During the extension period, those clamping jaws which hold the bone fragment to be transported are slightly loosened so that they can be displaced on the frame. The module permits a controlled displacement of the clamping jaws along the frame. This can be achieved by turning a hand wheel. The displacement is effected in particular by a threaded rod/spindle. After displacement of the clamping jaws by a certain distance (e.g. 0.25 mm), the clamping jaws are tightened again and, independently of the module, the force flows directly to the frame. The module is not used during the static period. After extension is complete, i.e. the bone has reached the correct anatomical length or the gap has been closed the module can be removed and the frame remains alone on the bone until the latter has consolidated. The design and operation of the fixation device 1 is now provided.

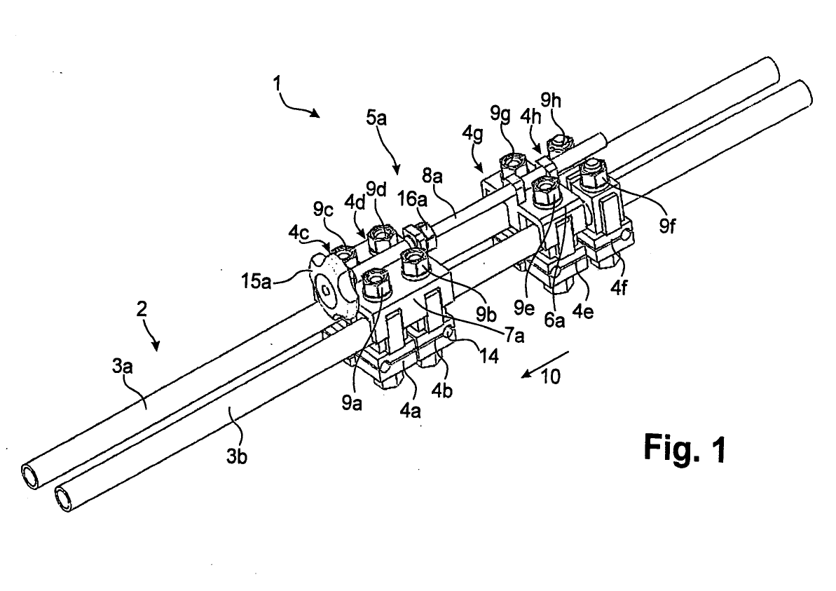

FIG. 1 shows a perspective diagram of a fixation device 1. The fixation device 1 includes a frame 2, two rods 3a, 3b, and a plurality of clamping jaws 4a-h attachably connected to the frame 2, and more precisely connected to the rods 3a and 3b. The rods may preferably be parallel to each other. However, other configurations of the rods, relative to each other, are contemplated. For example in FIG. 1, clamping jaws 4a, 4b, 4c, and 4d form one group of jaws, and 4e, 4f, 4g, and 4h are arranged to form another group of jaws. Each clamping jaw is connected to a connecting means, for example to a bone-retaining pin (not shown) which may be introduced into the bone. The clamping jaws permit connection of the bone to the rods 3a, 3b and hence to the frame 2. When the bone is cut through, the load is transmitted/transferred via the connecting means to the frame. Bore 14 of clamping jaw 4b, for example, may house the bone-retaining pin.

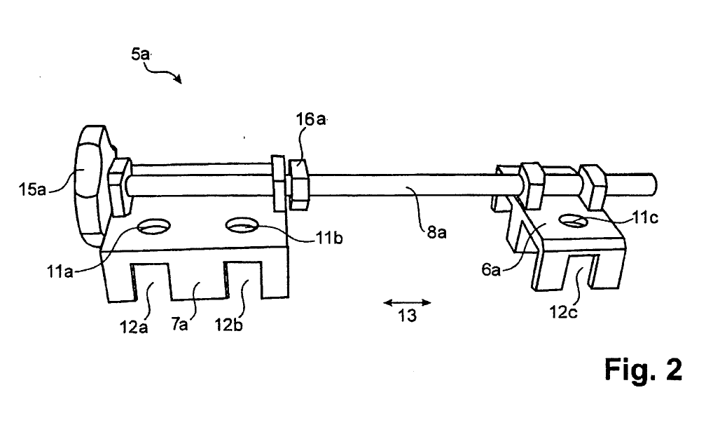

The fixation device may also include a module 5a. The module 5a is also shown schematically in FIG. 2. The module may be composed at least partly of a light metal, preferably of aluminum or of an aluminum alloy. In FIG. 1, the module 5a is connected to the frame 2. The module 5a has a first component 6a and a second component 7a. The first component 6a may be connected to the frame. This connection is particularly strong. The second component 7a on the other hand is displaceably connected to the frame. It is supported on the clamping jaws which are connected via the bone-retaining pins or connecting means to the bone fragment to be displaced.

FIG. 1 also shows that the first component 6a is preferably connected to the clamping jaws 4e and 4g. The connection of the first component 6a and of the second component 7a of the module 5a to the corresponding clamping jaw is preferably effected with the aid of the same screws and nuts 9a-h with which the clamping jaws 4 are also fixed on the rod 3a, 3b.

If it is intended to effect a displacement of a bone fragment, the nuts 9a-d are slightly undone, preferably by a half to a whole turn allowing the second component 7a to move. As a result, the clamping jaws 4a-4d and second component 7a are displaceable on the rods 3a, 3b. However, it should be noted that threaded rod 8a of the module 5a initially prevents this displacement. The threaded rod 8a performs a certain retaining function which proves very advantageous during the displacement phase.

For displacement of the bone fragment, the threaded rod 8a is rotated by turning the hand wheel 15a or with the aid of an automated/power tool such as an electric drive. The electric drive may have a timer and an incremental limiter so that displacement movement of the second component 7a can take place with automatic control. The means for which displacement is achieved may also have a readable display showing the displacement movement. It is also possible to provide a hexagon nut 16a, so that rotational movement is effected not by means of the hand wheel but by means of a corresponding tool. The hexagon nut 16a also may act as a stop measure and aid in preventing the second component 7a from returning to its original position until the nuts 9a-d are tightened. By rotating the threaded rod/spindle 8a, relative movement is produced, indicated by the arrow 10. The length of the displacement can be defined by the pitch of the threaded rod 8a. Thus, for example, a complete turn may correspond to a distance of 1 mm, which is a usual distance per day for a bone extension. It should be noted that the nuts 9e-9h are not undone during the extension process. They remain tight.

The diagram of FIG. 2 shows the design of the module 5a in detail. The module 5a may include a threaded rod/spindle. Both the first component and the second component have recesses 12a, 12b, 12c for engaging in each case one clamping jaw. The two components 6a, 7a are mounted on the clamping jaws. Furthermore, bores 11a, 11b, 11c are provided into which the screws (not shown) of the clamping jaws can be inserted. By means of this design, an accurate fit of the first component 6a and of the second component 7a on the clamping jaws may be achieved, as is evident from the diagram of FIG. 1. It should be noted that, in the diagram of FIGS. 1 and 2, the first component 6a engages only two clamping jaws 4e and 4g, whereas the second component 7a engages four clamping jaws 4a, 4b, 4c, 4d. As is easily evident to the person skilled in the art this design can be modified as desired without departing from the principle according to the invention.

From FIG. 2, it is furthermore clear that a movement of the components 6a and 7a relative to one another is possible in both directions, indicated by the arrow 13. In the working example of FIG. 1, the first component 6a is fixed whereas the second component 7a experiences a relative movement as a result of the movement of the threaded rod/spindle 8a, for example with the aid of the hand wheel 15a. Alternatively, however the component 6a can also be moved by arranging the component 7a in a fixed manner. Furthermore, the bone fragment could also be displaced in both directions if the component 6a is fixed. The direction of rotation on the spindle determines the displacement direction. The module 5a may preferably be mounted independent of the desired displacement direction of the bone fragment. It is therefore also distinguished by universality.

While the foregoing description and drawings represent the preferred embodiments, it will be understood that various additions, modifications and substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. The presently disclosed embodiments are therefore to be considered in all respects as illustrative and not restrictive, the scope of the invention being indicated by the appended claims, and not limited to the foregoing description.

What is claimed:

1. An external fixation device for osteosynthesis comprising:
a frame having at least one rod, the frame capable of connection to a bone via connecting members;
a plurality of clamping jaws, the plurality of clamping jaws connecting the connecting member to the frame; and
a module mounted on the frame, the module having a first component and a second component, the first component connected to the frame and the second component connected to at least one clamping jaw, and moving means to move the second component relative to the first component.

2. A fixation device according to claim 1, wherein the frame includes two rods arranged parallel to each other.

3. A fixation device according to claim 1, wherein the moving means comprises a threaded spindle.

4. A fixation device according to claim 1, wherein at least one of the first component and second component have at least one recess which can be mounted above one clamping jaw.

5. A fixation device according to claim 1, wherein at least one of the first component and second component have bores for the passage of a fixing member.

6. A fixation device according to claim 1, wherein the module is composed at least partly of a light metal.

7. A fixation device according to claim 1, wherein the moving means comprises an electric drive.

8. A fixation device according to claim 7, wherein the electric drive has a timer and an incremental limiter such that the displacement movement can take place with automatic control.

9. A fixation device according to claim 1, wherein the means has a readable display for the displacement movement.

10. A method of performing osteosynthesis comprising the steps of:
connecting a fixation device via connecting member to two bone fragments, the fixation device including a frame having at least one rod, a plurality of clamping jaws, the plurality of clamping jaws connecting the connecting member to the frame, and a module mounted on the frame, the module having a first component and a second component, the first component connected to the frame and the second component connected to at least one clamping jaw, and moving means comprising a hand wheel and threaded rod to move the second component relative to the first component; and
displacing a bone fragment relative to another by loosening the clamping jaws which hold the bone fragment to be transported such that the clamps can be displaced on the frame by turning the hand wheel to rotate the threaded rod, permitting a controlled displacement of the clamping jaws along the frame.

11. A method according to claim 10, wherein osteosynthesis is performing in alternating extension and static periods, the extension period lasting two minutes and the static period lasting six hours.

12. A method according to claim 10, further comprising the step of tightening all clamping jaws of the fixation device during a static period, wherein the module is ineffective and not under mechanical load.

13. A fixation device comprising:
a frame having an elongated rod; and
a module having a first component and a second component mounted and guidable along the elongated rod,
wherein the module is clamped to and releasable from the elongated rod.
